(19) United States
(12) Patent Application Publication
(10) Pub. No.: US 2013/0231287 A1
(43) Pub. Date: Sep. 5, 2013

(54) PEGYLATED ALBUMIN POLYMERS AND USES THEREOF

(71) Applicants: Parimala Nacharaju, Staten Island, NY (US); Marcos Intaglietta, La Jolla, CA (US)

(72) Inventors: Parimala Nacharaju, Staten Island, NY (US); Marcos Intaglietta, La Jolla, CA (US)

(21) Appl. No.: 13/578,684

(22) PCT Filed: Feb. 22, 2011
(86) PCT No.: PCT/US11/00325

§ 371 Date: Feb. 12, 2013

Related U.S. Application Data
(60) Provisional application No. 61/339,020, filed on Feb. 25, 2010.

(51) Int. Cl. C07K 14/76

(52) U.S. Cl. CPC C07K 14/76
USPC 514/15.2; 530/369; 428/402

(57) ABSTRACT

The present invention provides an albumin polymer and a process of making an albumin polymer, the process comprising breaking instrinsic disulfide bridges in albumin with a reducing agent and crosslinking inter- and intra-molecular disulfide bridges so as to form the polymer. The present invention also provides PEGylated albumin polymers and methods of making PEGylated albumin polymers, the methods comprising polymerizing albumin by crosslinking inter- and intra-molecular disulfide bridges, and PEGylating the albumin polymer. The present invention further provides a pharmaceutical composition useful as a blood plasma expander, blood substitute or for drug delivery, and methods of treating blood loss in a subject and of delivering drugs to a subject's tissue, the pharmaceutical composition comprising a therapeutically effective amount of the PEGylated albumin polymer.

### CROSS-REFERENCE TO RELATED APPLICATIONS

This application claims benefit of U.S. Provisional Application No. 61/339,020, filed Feb. 25, 2010, the contents of which are hereby incorporated by reference.

### FIELD OF THE INVENTION

The present invention relates generally to PEGylated albumin polymers and their uses for enhanced plasma expansion and drug delivery.

### BACKGROUND OF THE INVENTION

Throughout this application various publications are referred to by number in parenthesis. Full citations for these references may be found at the end of the specification. The disclosures of these publications are hereby incorporated by reference in their entirety into the subject application to more fully describe the art to which the subject invention pertains.

Plasma expansion is the initial treatment for blood losses and is a continued treatment in patients with prolonged recovery. Blood loss is associated with conditions such as trauma and surgery. Plasma expansion treatment for blood loss is often a critical treatment for combat casualties, victims of highway accidents and causalities in remote areas.

Plasma expansion is a means for restitution or maintenance of intravascular blood volume, often accomplished by the transfusion of blood in order to maintain oxygen carrying capacity and to correct blood losses. During the past two decades, the technology has focused on combining plasma expansion with oxygen transport and delivery to tissue. This approach has shown to be a moving target, with uncertain outcome, as academic and industrial efforts continually fail to deliver an oxygen-carrying plasma expander or “blood substitute”(1).

Providing oxygen transport capacity to a plasma expander fails due to problems intrinsic to the oxygen carrier. Two materials that increase plasma oxygen carrying capacity are perfluorocarbons (PFBs) and cell free hemoglobin (CFH). PFBs are not water soluble and must be emulsified (4). Hemoglobin (Hb) outside of the red blood cell environment is inherently toxic (2, 3). Masking toxicity by conjugation with other molecular species in protecting molecular constructs such as polyethylene glycol (PEG), toxicity emerges when the organism presents components of endothelial dysfunction (prevalent in at least 10% of the young healthy population). Hb can be encapsulated in various vesicle like systems, however these cannot exceed a particle diameter of 200 nm, in order to prevent immunological and inflammatory particle size dependant cardiovascular responses (5). This limitation causes the encapsulation ratio for both PFBs and Hb vesicles (with PEG conjugation) at best to be 30-70% efficient (i.e., 30% of the material is encapsulation related), a significant load for the organism.

It has been determined that the maintenance of microvascular function, and in particular capillary perfusion or functional capillary density (“FCD”, i.e. the number of capillaries perfused with passing red blood cells per unit tissue area) far outweighs the need for maintaining intrinsic oxygen carrying capacity, even though both functions may be considered at times linked, but are not (6). Maintenance and improvement of microvascular function requires 1) institution of blood fluid biophysical properties that enhance microvascular function, and 2) avoidance of microvascular failure rooted in endothelial dysfunction and inflammation.

Studies with animal models of extreme hemodilution, hemorrhagic shock and endotoxemia indicate that the complications of these conditions are due to the reduced microvascular function as a result of the lowering of functional capillary density (FCD), rather than the declined oxygen-carrying capacity of the blood (6). The blood viscosity tightly regulates microvascular flow homeostasis and if reduced below a critical level will lead to reduced stimulation and malfunction of the endothelium, causing capillary collapse and reduced tissue perfusion. Increasing blood viscosity to normal levels using viscous plasma expanders has been shown to restore FCD and microvascular function in these models. Viscogenic colloids such as dextrans and alginates maintain FCD significantly. However, these colloids can only be used at comparatively low hematocrits (<18%) because a higher concentration leads to red blood cell aggregation.

Plasma expanders currently in use include albumin, Pentaspan®, Hextend® and dextran. These products have a short circulation life and cause adverse effects such as red blood cell aggregation and interference with blood coagulation. Some plasma expanders contained modified Hb. Although the toxicity of molecular Hb can be compensated for by conjugation with PEG, it cannot be completely eliminated (7). However, PEG-Hb has been demonstrated in many experimental studies to be an exceptional plasma expander able to maintain FCD in hemorrhage, acute anemia, and endotoxemia far better than all other conventional plasma expanders.

PEGylated albumins serve as excellent plasma expanders in hemorrhagic shock and endotoxemia induced hamster models. PEGylated albumin theoretically remains in the intravascular compartment for a longer time than the non-PEGylated albumin, providing larger and longer lasting plasma volume expansion for identical infused volumes. However, they are not ideal since PEGylated albumins are associated with high colloid oncotic pressure (COP) in addition to high viscosity (8). The high COP causes diffusion of interstitial fluid into vasculature thus reducing the plasma viscosity. PEGylation increases the COP and viscosity of proteins in parallel. All PEGylated albumins and PEGylated hemoglobins designed so far are associated with moderate viscosity as well as moderate COP.

The current invention solves this problem with the design of PEG-albumin polymers with high viscosity and low COP that serve as optimal plasma expanders.

### SUMMARY OF THE INVENTION

A process for preparing an albumin polymer, the method comprising contacting albumin with a reducing agent under conditions causing dissociation of intrinsic albumin inter-molecular disulfide bridges and subsequently permitting crosslinking of the albumin by formation of new inter-molecular and intra-molecular disulfide bridges, so as to form the albumin polymer.

An albumin polymer comprising one or more non-intrinsic crosslinking inter-molecular and intra-molecular disulfide bridges.

A process for preparing a PEGylated albumin polymer, the method comprising contacting an albumin polymer with a derivatized polyethylene glycol (PEG) under conditions permitting formation of a bond between the PEG and the albumin polymer so as to form a PEGylated albumin polymer.

A PEGylated albumin polymer prepared by any of the instant processes.

A pharmaceutical composition comprising a therapeutically effective amount of any of the instant PEGylated albumin polymers in a pharmaceutically acceptable carrier.

A method of treating blood loss in a subject, the method comprising administering to the subject any of the instant PEGylated albumin polymers or compositions containing such, in a therapeutically effective amount so as to treat the blood loss.

A method of delivering drugs to a subject's tissue, the method comprising administering to the subject any of the instant PEGylated albumin polymers bound to at least one drug molecule in a therapeutically effective amount.

Use of any of the instant PEGylated albumin polymers for treatment of blood loss in a subject.

Use of any of the instant PEGylated albumin polymers as a drug delivery vehicle.

The present invention provides a method of preparing albumin polymer, the method comprising polymerizing albumin by crosslinking inter- and intra-molecular disulfide bridges. The present invention also provides an albumin polymer prepared by crosslinking inter- and intra-molecular disulfide bridges.

The present invention further provides a method of preparing a PEGylated albumin polymer, the method comprising polymerizing albumin by crosslinking inter- and intra-molecular disulfide bridges, and PEGylating the albumin polymer. The present invention additionally provides a PEGylated albumin polymer prepared by polymerizing albumin by crosslinking inter- and intra-molecular disulfide bridges, and PEGylating the albumin polymer.

The present invention provides a pharmaceutical composition useful as a blood plasma expander, blood substitute or for drug delivery, the pharmaceutical composition comprising a therapeutically effective amount of the PEGylated albumin polymer prepared by polymerizing albumin by crosslinking inter- and intra-molecular disulfide bridges, and PEGylating the albumin polymer, in a pharmaceutically acceptable carrier.

The present invention also provides a method of treating blood loss in a subject, the method comprising administering the PEGylated albumin polymer prepared by polymerizing albumin by crosslinking inter- and intra-molecular disulfide bridges, and PEGylating the albumin polymer, in a therapeutically effective amount.

The present invention further provides a method of delivering drugs to a subject's tissue, the method comprising administering the PEGylated albumin polymer prepared by polymerizing albumin by crosslinking inter- and intra-molecular disulfide bridges, bound to at least one drug molecule in a therapeutically effective amount.

### BRIEF DESCRIPTION OF THE DRAWINGS

FIG. 1A-1C. (1A) Direct PEGylation of protein using succinimidyl chemistry. (1B) Direct PEGylation with cyanuric chloride-PEG. (1C) Extension Arm Facilitated PEGylation using thiolation reagent and maleimide-PEG

FIG. 2. Size exclusion chromatography of albumin polymer. The reaction mixture of albumin polymer displays two peaks, one eluting at the position of albumin (71 min) and the other eluting much earlier (35 min), corresponding to albumin polymer.

FIG. 3. Acute Hemodiltuion: FCD as a function of plasma viscosity. High viscosity solutions and PEG materials maintain FCD better than low viscosity solutions. HCT= 11 %. Dex 70 and 500, 70 and 500 kDa, MPA, PEG-Alb 4% and MP4, PEGHb 4% (Sangart, San Diego, Calif.); PBH, Polymerized bovine Hb (Biopure, Boston Mass.). HbV, Hb vesicles. (n=5, mean±SEM) (9-11).

FIG. 4. Functional capillary density attained during the resuscitation from hemorrhagic shock with different oxygen carrying and non carrying colloidal solutions as a function of the plasma viscosity. It is apparent that the higher plasma viscosities uniformly improve microvascular function independently whether the material carries oxygen. The line shown is indicative of the trend of the data. HES, hydroxyethyl starch, HbV, Hb encapsulated vesicles; PEG-Hb, PEG conjugated Hb; PEG-Alb, PEG conjugated albumin, letters correspond to references: a(1); b (2); c (3); d (4); e (5); f (6); g (7).

FIG. 5. Binding of warfarin (WF) to albumin and PEG-albumin-polymer at varying micromolar concentrations of the drug.

FIG. 6. Kinetics of DTT induced Polymerization of human serum albumin (HAS). HSA (0.5 mM) was incubated with DTT in PBS at room temperature and the absorbance of the reaction mixture at 700 nm was recorded.

FIG. 7. Monitoring the formation of HSA polymer by size exclusion chromatography. HSA (0.5 mM) was incubated with 50 mM DTT in PBS at room temperature. Aliquots of reaction mixture were taken out at different time intervals, diluted 4 times and analyzed on Superose-12. Peak 1 and 2 correspond to the monomer and dimer of HSA, respectively. Peak 3 is the polymer/oligomer of HSA. The vertical line drawn along Peak 3 indicates the shift of the position of Peak 3 with the time of incubation.

FIG. 8. Cross-linking of proteins. Maleimide group is added on amino group of protein 1 with MS reagent and thiol group is added on amino group of protein 2 with IT. Mixing of these two proteins generates a maleimide-thiol cross-link between the two proteins.

### DETAILED DESCRIPTION OF THE INVENTION

A process for preparing an albumin polymer, the method comprising contacting albumin with a reducing agent under conditions causing dissociation of intrinsic albumin inter-molecular disulfide bridges and subsequently permitting crosslinking of the albumin by formation of new inter-molecular and intra-molecular disulfide-bridges, so as to form the albumin polymer.

In an embodiment, the reducing agent is dithiothreitol or tris(2-carboxyethyl)phosphine. In an embodiment, the process further comprises contacting the albumin polymer with a derivatized polyethylene glycol (PEG) under conditions permitting formation of a bond between the PEG and the albumin polymer. In an embodiment, the derivatized PEG is succinimidyl-PEG, cyanuric chloride-PEG or maleimide-PEG. In an embodiment, the process further comprises purifying the albumin polymer by size-exclusion chromatography prior to PEGylating the albumin polymer.

An albumin polymer comprising one or more non-intrinsic crosslinking inter-molecular and intra-molecular disulfide bridges.

A process for preparing a PEGylated albumin polymer, the method comprising contacting an albumin polymer with a derivatized polyethylene glycol (PEG) under conditions permitting formation of a bond between the PEG and the albumin polymer so as to form a PEGylated albumin polymer.

In an embodiment, a reducing agent is used to dissociate one or more intrinsic disulfide bonds of the albumin before polymerizing the albumin. In an embodiment, the reducing agent is dithiothreitol or tris(2-carboxyethyl)phosphine. In an embodiment, the method further comprising separating the polymerized albumin from unreacted albumin before PEGylation. In an embodiment, the polymerized albumin is separated by size exclusion chromatography. In an embodiment, the derivatized PEG is succinimidyl-PEG, cyanuric chloride-PEG or maleimide-PEG. In an embodiment, the method of PEGylating the albumin polymer comprises:
a) contacting the albumin polymer with a thiol agent; and
b) contacting the product of step a) with maleimide-PEG,
   so as to thereby form a PEGylated albumin polymer.

In an embodiment, the process further comprises bonding at least one albumin monomer to the surface of the PEGylated albumin polymer. In an embodiment, the process further comprises bonding of at least one albumin monomer to the surface of the PEGylated albumin polymer is effected through a maleimide-thiol reaction.

A PEGylated albumin polymer prepared by any of the instant processes. In an embodiment the PEGylated albumin polymer has a hydrodynamic radius of between 25 and 200 nm. In an embodiment the PEGylated albumin polymer has a hydrodynamic radius of between 60 and 100 nm. In an embodiment the PEGylated albumin polymer has a hydrodynamic radius of between 60 and 80 nm. In an embodiment the PEGylated albumin polymer has a viscosity between 5 and 15 centipoise (cP) at 2.6% protein concentration. In an embodiment the PEGylated albumin polymer has a viscosity between 7 and 10 cP at 2.6% protein concentration. In an embodiment the PEGylated albumin polymer has a viscosity of 8.3 cP at 2.6% protein concentration. In an embodiment the PEGylated albumin polymer has a colloid osmotic pressure between 0 and 60 mm Hg at 2.6% protein concentration. In an embodiment the PEGylated albumin polymer has a colloid osmotic pressure between 40 and 50 mm Hg at 2.6% protein concentration. In an embodiment the PEGylated albumin polymer has a colloid osmotic pressure of 44 mm Hg at 2.6% protein concentration. In an embodiment the PEGylated albumin polymer at 2.6% protein concentration does not elicit red blood cell aggregation in a human subject at hematocrits of 10%, 18%, 20%, 25% or 30%.

A pharmaceutical composition comprising a therapeutically effective amount of any of the instant PEGylated albumin polymers in a pharmaceutically acceptable carrier.

In an embodiment at least one drug molecule is bound to the PEGylated albumin polymer. In an embodiment at least one albumin monomer is bound to the surface of the PEGylated albumin polymer. In an embodiment the pharmaceutical composition is formulated for intravenous administration.

A method of treating blood loss in a subject, the method comprising administering to the subject any of the instant PEGylated albumin polymers or compositions containing such, in a therapeutically effective amount so as to treat the blood loss. In an embodiment the method comprises administration of a PEGylated albumin polymer, which PEGylated albumin polymer does not elicit red blood cell aggregation at 2.6% protein concentration in a human subject at hematocrits of 10%, 18%, 20%, 25% or 30%.

In an embodiment the administration is intravenous. In an embodiment the method results in an amelioration of the clinical impairment or symptoms of the subject's blood loss.

A method of delivering drugs to a subject's tissue, the method comprising administering to the subject any of the instant PEGylated albumin polymers bound to at least one drug molecule in a therapeutically effective amount.

In an embodiment the administration is intravenous. In an embodiment the surface of the PEGylated albumin polymer is decorated with albumin monomers.

Use of any of the instant PEGylated albumin polymers for treatment of blood loss in a subject.

Use of any of the instant PEGylated albumin polymers as a drug delivery vehicle.

The present invention provides a method of preparing albumin polymer, the method comprising polymerizing albumin by crosslinking inter- and intra-molecular disulfide bridges. The present invention also provides an albumin polymer prepared by crosslinking inter- and intra-molecular disulfide bridges.

Albumin polymers can be created by dissociating albumin's intrinsic (i.e. naturally occurring) disulfide bonds and allowing the resultant free thiols to form new inter- and intra-molecular disulfide bridges. Any reducing agent known in the art can be used. For example, dithiothreitol (DTT) or tris(2-carboxyethyl)phosphine (TCEP) can be used. Polymerizing albumin via thiol modification leaves the surface amino groups of the albumin polymer available for further derivation or PEGylation.

The present invention further provides a method of preparing a PEGylated albumin polymer, the method comprising polymerizing albumin by crosslinking inter- and intra-molecular disulfide bridges, and PEGylating the albumin polymer. As used herein, “PEGylation” means linking to polyethylene glycol (PEG), and a “PEGylated” albumin is an albumin that has PEG conjugated to it. The present invention additionally provides a PEGylated albumin polymer prepared by polymerizing albumin by crosslinking inter- and intra-molecular disulfide bridges, and PEGylating the albumin polymer.

Each PEG chain may have a molecular weight of 200 daltons to 20,000 daltons, preferably 3,000 to 5,000 daltons, and more preferably 5,000 daltons. PEGs of various molecular weights, conjugated to various groups, can be obtained commercially, for example from NOF America, Lysan Bio, Inc., and SunBio, Inc.

After polymerization and before PEGylation, polymerized albumin can be separated from the unreacted albumin by any method known in the art including, but not limited to, size exclusion chromatography.

Albumin polymers can be PEGylated by any method known in the art including, but not limited to, succinimidyl chemistry, or cyanuric chloride-PEG or maleimide-PEG in the presence of a thiolation reagent (extension arm facilitated PEGylation). Any succinimidyl-PEG reagent known in the art can be used for the PEGylation of albumin polymer.

Albumin has two high affinity drug binding sites. Therefore, non-covalent bonding and covalent attachment of drugs can be effected. Additionally, combination therapy can be achieved with more than one drug employing covalent and non-covalent interactions. PEGylated albumin polymers have a lower binding affinity than that of non-PEGylated albumin. Any drug molecule known in the art can be attached to the PEGylated albumin polymer by any method known in the art, such as by covalently or non-covalently attaching the drug molecule to amino groups on the PEGylated albumin polymer. The drug-carrying PEGylated albumin polymer has an increased circulation life and can enhance pharmacokinetics of the drug. Albumin receptors are widely distributed in tissue and organs such as the liver, lungs, and intestines, allowing receptor-mediated delivery of drugs bound to albumin. To increase albumin receptor recognition of PEGylated albumin polymers, albumin monomers can be bonded to the surface of the PEGylated albumin polymer. The PEGylated albumin polymer can be surface-decorated with one or more albumin monomers by any method known in the art including, but not limited to, conjugating albumin monomer(s) to the surface of the nanoparticles via maleimide-thiol reactions.

The molecular size of the PEGylated albumin polymer can range from 25 to 200 nm, depending on the extent of polymerization and PEGylation. More preferably, the molecular size of the PEGylated albumin polymer ranges between 60 and 100 nm. Most preferably, the molecular size of the PEGylated albumin polymer ranges between 60 and 80 nm. The molecular size differs depending on the extent of polymerization and PEGylation. The size of the PEGylated albumin polymer allows for extended circulation life, resulting in larger and longer lasting plasma volume expansion for an identical amount of administered PEGylated albumin. Additionally, the size of the PEGylated albumin polymer extends circulation time, reducing clearance of the drug bound to the PEGylated albumin polymer and allowing for less frequent administration of the drug. Molecular size can be determined by any method known in the art, for example, matrix-assisted laser desorption/ionization (MALDI) mass spectrometry, NMR, or dynamic light scattering.

An ideal blood plasma expander or blood substitute should have a high viscosity and low colloid osmotic pressure. A high viscosity results in a larger plasma expansion for the same volume of PEGylated albumin polymer administered while a high colloid osmotic pressure results in a diffusion of interstitial fluid into the vasculature, reducing the plasma viscosity. The viscosity of the PEGylated albumin polymer can range from 5 to 15 centiPoise (cP). Preferably, the viscosity of the PEGylated albumin polymer is between 7 and 10 cP. More preferably, the viscosity of the PEGylated albumin polymer is 8.3 cP. Viscosity is measured at 2.6% PEGylated albumin polymer concentration. The colloid osmotic pressure can range between 0 and 60 mm Hg. Preferably, the colloid osmotic pressure ranges between 40 and 50 mm Hg. More preferably, the colloid osmotic pressure is 44 mm Hg. Colloid osmotic pressure is measured at 2.6% PEGylated albumin polymer concentration.

The present invention provides a pharmaceutical composition useful as a blood plasma expander, blood substitute or for drug delivery, the pharmaceutical composition comprising a therapeutically effective amount of the PEGylated albumin polymer prepared by polymerizing albumin by crosslinking inter- and intra-molecular disulfide bridges, and PEGylating the albumin polymer, in a pharmaceutically acceptable carrier.

The present invention also provides a method of treating blood loss in a subject, the method comprising administering the PEGylated albumin polymer prepared by polymerizing albumin by crosslinking inter- and intra-molecular disulfide bridges, and PEGylating the albumin polymer, in a therapeutically effective amount.

The present invention further provides a method of delivering drugs to a subject's tissue, the method comprising administering the PEGylated albumin polymer prepared by polymerizing albumin by crosslinking inter- and intra-molecular disulfide bridges, bound to at least one drug molecule in a therapeutically effective amount.

The pharmaceutically acceptable carrier must be compatible with the PEGylated albumin nanoparticles, and not deleterious to the subject. Examples of acceptable pharmaceutical carriers include carboxymethylcellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage and may be prepared by any method known in the pharmaceutical art. For example, PEGylated albumin polymer may be brought into association with a carrier or diluent, as a suspension or diluent. Optionally, one or more accessory ingredient, such as buffers, flavoring agents, surface active ingredients, and the like may also be added. The choice of carriers will depend on the method of administration. The pharmaceutical composition would be useful for administering PEGylated albumin polymer as a blood plasma expander and blood substitute or for drug delivery. These amounts may be readily determined by one in the art. In one embodiment, the PEGylated albumin polymer is the sole active pharmaceutical ingredient in the formulation or composition. In another embodiment, there may be a number of active pharmaceutical ingredients in the formulation of composition aside from the PEGylated albumin polymer. In this embodiment, the other active pharmaceutical ingredients in the formulation or composition must be compatible with the PEGylated albumin particle.

The PEGylated albumin polymer can be administered in a formulation or pharmaceutical composition comprising the PEGylated albumin polymer. Further, the PEGylated albumin polymer can be administered in a formulation or pharmaceutical composition consisting essentially of the PEGylated albumin polymer. Additionally, the PEGylated albumin polymer may also be administered in a formulation or pharmaceutical composition where the pharmaceutically active agent consists of the PEGylated albumin polymer.

In the present invention, the PEGylated albumin polymer is administered to a subject for treatment of a subject's blood loss or in order to deliver drugs to a subject's tissue in an amount and manner which is effective to treat a subject's blood loss or to deliver drugs to a subject's tissue, respectively. “Effective to treat” as used herein means effective to ameliorate or minimize the clinical impairment or symptoms of a subject's blood loss. “Effective to deliver” as used herein means effective to deliver a clinically significant amount of drug to a subject's tissue. A “clinically significant” amount of drug means an amount of drug effective to effect a clinically significant change in a subject's tissue. The PEGylated albumin polymer may be used to deliver many types of drugs including, but not limited to: penicillin; sulfonamides; indole compounds; benzodiazapines; hydrophobic drugs that otherwise require detergents, for example, paclitaxel; or ions such as copper II, nickel II, calcium II, or zinc II. The amount of PEGylated albumin polymer effective to treat a subject's blood loss or deliver drugs to a subject's tissue will vary depending on the condition, the clinical severity of the condition, and the PEGylated albumin polymer used. Appropriate amounts of PEGylated albumin polymer effective to treat a subject's blood loss or to deliver drugs to a subject's tissue can be readily determined by the skilled artisan without undue experimentation. Additionally, the manner of administration of PEGylated albumin polymer which is effective to treat a subject's blood loss depends on the severity of the blood loss and the subject's overall condition, among other factors. The manner of administration of PEGylated albumin polymer which is effective to deliver drugs to a subject's tissue depends on the tissue involved. When the PEGylated albumin polymer is being used to treat a subject's blood loss, the PEGylated albumin polymer must be administered in a manner that allows the subject's blood plasma volume to be expanded without harming the subject's vasculature. When the PEGylated albumin polymer is being used to deliver drugs to a subject's tissue, the PEGylated albumin polymer is preferably administered in a manner that allows the drug to reach the involved tissue without harming the subject.

The PEGylated albumin polymer is to be administered to a subject in an amount and manner effective to treat the subject's blood loss or deliver drugs to the subject's tissue. According to the method of the present invention, the PEGylated albumin polymer may be administered to a subject by any known procedure including, but not limited to, parenteral administration, oral administration, transdermal administration, and administration through an osmotic mini-pump. Preferably, the PEGylated albumin polymer is administered parenterally, such as intravenously or by injection. Preferably, administration by injection comprises administration by injection into the subject's vasculature.

For a parenteral administration, the PEGylated albumin polymer may be combined with a sterile aqueous solution which is preferably isotonic with the blood of the subject, unless the subject also requires a therapy to alter tonicity, in which case the appropriate tonicity can be used. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be present in unit or multi-dose containers, such as sealed ampoules or vials. The formulation may be delivered by any mode of injection, including, without limitation, epifascial, intrasternal, intravascular, intravenous, parenchymatous, or subcutaneous. The formulation may additionally be provided in dried form for reconstitution by the administrator of the PEGylated albumin polymer. Such a dried form permits easier shipment and storage of the PEGylated albumin polymer reducing risks from storage and breakage. The dried formulation provides ease-of-use to the PEGylated albumin polymer in arenas such as combat casualties, third-world countries, and casualties in remote areas. The dried formulation can be reconstituted by combination with a sterile aqueous solution or other carrier which is pharmaceutically acceptable for parenteral administration.

For oral administration, the formulation of the PEGylated albumin polymer may be presented as capsules, tablets, powder, granules, or as a suspension. The formulation may have conventional additives, such as lactose, mannitol, corn starch, or potato starch. The formulation may also be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For transdermal administration, the PEGylated albumin polymer may be combined with skin penetration enhancers, such as propylene glycol, polyethylene glycol, isopropanol, ethanol, oleic acid, N-methylpyrrolidone, and the like, which increase the permeability of the skin to the pyruvate or pyruvate derivative, and permit the pyruvate or pyruvate derivative to penetrate through the skin and into the bloodstream. The pyruvate or pyruvate derivative/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to backing material to provide a patch.

The PEGylated albumin polymer of the present invention also may be released or delivered from an osmotic mini-pump. The release rate from an elementary osmotic mini-pump may be modulated with a microporous, fast-response gel disposed in the release orifice. An osmotic mini-pump would be useful for controlling the release of, or targeting delivery of, PEGylated albumin polymer delivery.

The present invention provides the use of the PEGylated albumin polymer prepared by polymerizing albumin by crosslinking inter- and intra-molecular disulfide bridges, as a blood plasma expander or blood substitute. The present invention also provides the use of the PEGylated albumin polymer prepared by polymerizing albumin by crosslinking inter- and intra-molecular disulfide bridges, as a drug delivery vehicle.

Where a numerical range is provided herein, it is understood that all numerical subsets of that range, and all the individual integers contained therein, are provided as part of the invention. Thus, from 40 to 50 mm Hg colloid osmotic pressure includes the subset of 40-45 mm Hg, the subset of primers which are 41-49 mm Hg etc. as well as 40 mm Hg, 41 mm Hg, 42 mm Hg, etc. up to and including 50 mm Hg.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

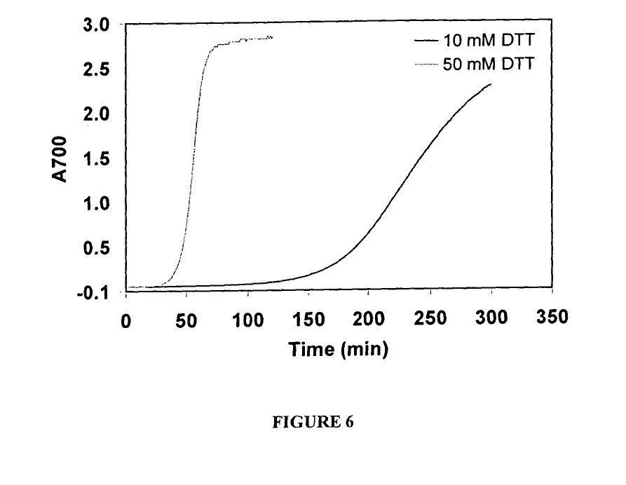

Polymerization of albumin: Human serum albumin (HSA) (0.5 mM) was incubated with 10 or 50 mM dithiothreitol (DTT) at room temperature (25° C.) and the kinetics of polymerization were monitored by recording the absorbance of the reaction mixture at 700 nm (FIG. 6). After a time lag, the absorbance of the reaction mixture increased indicating the formation of insoluble polymer. Once the insoluble polymer formed, the absorbance increased rapidly. This increase was more dramatic at the higher concentration of DTT (50 mM) and finally, the curve plateaued. This polymerization pattern resembles nucleation-dependent polymerization of sickle cell hemoglobin.

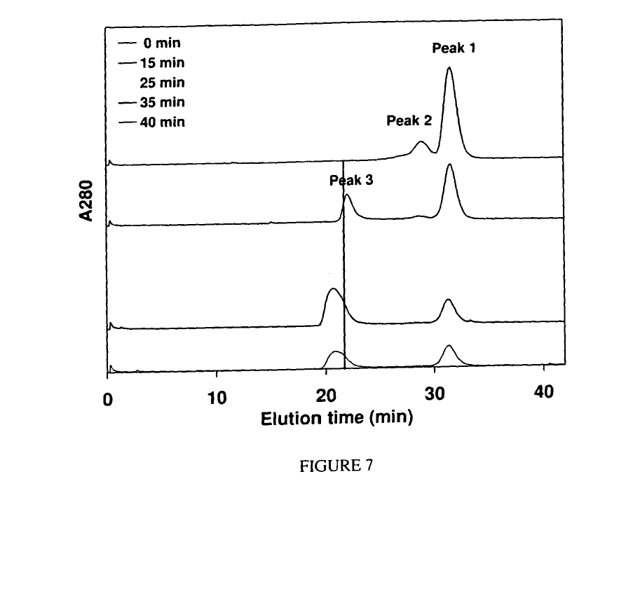

At different time intervals, aliquots of the reaction mixture were taken out, diluted fourfold and centrifuged at 16,000 g for 5 minutes. The supernatants were analyzed by size exclusion chromatography for HSA polymers (FIG. 7). In each chromatogram, only two peaks appeared, one corresponding to the HSA polymer/oligomer (early eluting) and the other related to the monomer (eluting later). The early time points (before increase in the absorbance of the reaction mixture at 700 nm) displayed the formation of soluble HSA polymers. With time, the size of the polymer increased as reflected by the reduction in the elution time of the polymer on SEC. The appearance of only one peak for the polymer and its symmetry, irrespective of the size of the polymer (elution time), indicated the homogeneity of the size of the polymer, and may also indicate the structural determinants that control the thiol-mediated polymerization aspects of HSA.

The time points corresponding to the increase in the absorbance of the reaction mixture at 700 nm, displayed reduced amounts of the polymer demonstrating the partitioning of the polymer into. insoluble phase. These aliquots still contained HSA monomer indicating that the formation of insoluble polymer from the soluble polymer is faster than the initial polymerization of monomer. This is consistent with the time lag and the dramatic increase in the absorbance of the reaction mixture at 700 nm.

The stability of the crosslinks of the polymer in plasma is tested by incubating the polymer (about 1%) in plasma (50%) at 37° C. for 24 hours. The sample was analyzed on size exclusion chromatography (SEC) before and after incubation. No difference in the chromatographic pattern was observed, demonstrating the stability of the polymer in plasma at 37° C.

Human serum albumin (HSA) plasma expansion property determination: the HSA polymer preparation containing the largest soluble polymer was selected. HSA (0.5 mM) was polymerized in the presence of 50 mM DTT in phosphate-buffered saline (PBS) at room temperature for 35 minutes and then half-diluted with 100 mM N-ethyl maleimide to block the remaining free thiols. The incubation was continued for 30 min at room temperature and then the sample was dialyzed with PBS at 4° C. overnight. This preparation contained about 50% of HSA polymer and 50% of monomer. This polymer can be purified to 100% homogeneity by removing the unreacted albumin using size exclusion chromatography.

PEGylation of HSA polymer: To further increase the viscosity of the polymer, the albumin polymer was PEGylated with SPA-PEG-5000 as a model PEG reagent. Alb-Polymer-50 (0.25 mM) was incubated with 10 mM SPA-PEG-5000 in PBS at 4° C., overnight. The unreacted PEG reagent was removed from the sample by diafiltration (Minim™, PALL Biopharmaceuticals, Port Washington, N.Y.), using a 50K membrane. This PEG-Alb-polymer-50 has a COP of 44 mmHg and a viscosity of 8.3 cp at 2.6% protein concentration. At the same concentration, a preparation of PEG-Albumin exhibited COP comparable to that of PEG-Alb-polymer-50. However, the viscosity of this sample was three-fold lower than that of the PEG-Alb-polymer-50. Another succinimidyl-PEG reagent with longer carbon chain, Sunbright® ME-050HS (NOF America, White Plains, N.Y.) also yielded similar results. HSA-polymer PEGylated by Maleimide-PEG-5000 (Lysanbio, Inc., Arab, Ala.) in the presence of 2-iminothiolane as well as Cycnuric chloride-PEG-5000 (Sigma, St. Louis, Mo.) also exhibited high viscosity and low COP.

Thus, any succinimidyl-PEG reagent can be used for the PEGylation of albumin polymer. Cyanuric chloride-PEG and maleimide-PEG in the presence of a thiolation reagent can also be used to add PEG.

The stability of the inter-molecular crosslinks of the polymer in plasma is tested by incubating the polymer (about 1%) in plasma (50%) at 37° C. for 24 h. The sample was analyzed on SEC before and after incubation. No difference in the chromatographic pattern was observed, demonstrating the stability of the polymer in plasma at 37° C. The free thiol content of the polymer is determined to be negligible (much below than one).

The PEG-Alb-Polymer did not induce red blood cell (“RBC”) aggregation in normal hamsters when introduced as a 10% blood volume (estimated as 7% of the body weight) hypervolemic bolus infusion.

The molecular size of the PEGylated albumin polymers has been determined by dynamic light scattering (Table 1). The size varied between 60 to 80 nm depending on the extent of polymerization and/or PEGylation. Preparations containing insoluble polymers exhibited larger hydrodynamic radius (about 100 nm). Thus, customization of size of the polymers is possible.

TABLE 1
Hydrodynamic radius (Rₕ) of albumin

| Sample | Rₕ (nm) |
|---|---|
| HSA | 4 |
| PEG-HSA | 8-10 |
| PEG-HSA-Polymer | 60-80 |

Surface decoration of albumin nanoparticles with albumin: Albumin monomers can be conjugated to the surface of nanoparticles to increase recognition of the nanoparticle by albumin receptors. This can also improve the drug carrying capability of the complex.

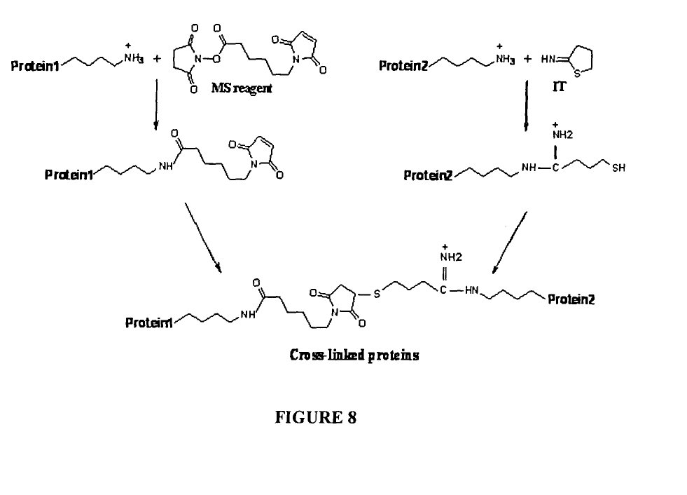

Maleimide-thiol reactions will be used for adding albumin on the surface of nanoparticles. Bi-functional reagents (“MS reagents”) carrying maleimide group and succinimidyl-active ester are commercially available. The active ester reacts with amino group of proteins (FIG. 8). The maleimide group present on the other end of the reagent can react with a thiol group with very high specificity.

Thiolation reagents such 2-iminothiolane (IT) can be used to add thiols on a protein. The amidine group of IT reacts with amino group of a protein and adds a thiol group at the distal end (FIG. 8). Therefore, generating thiols on one protein and adding maleimide groups on another protein is an excellent strategy to generate cross-links between proteins. The site-specificity of these cross-links is dictated by the reactivity of amino groups of proteins toward the succinimidyl-active ester of MS reagent and the amino-reactive group of the thiolation reagent.

MS reagents are available with varying length of alkyl chain that links the maleimide group and succinimidyl-active ester (Table 2). Similarly, thiolation reagents with varying spacer arm are also commercially available. This provides a wide range of spacing between the molecular surfaces of the crosslinking proteins. This feature can help to add multiple copies of bulky albumin molecules on the surface of albumin-nanoparticles.

TABLE 2
Reagents for protein crosslinking

| | | |
|---|---|---|
| | | Spacer |
| Reagent | Reagent Full Name | arm |

Reagents to add maleimides on protein amino groups

| | | |
|---|---|---|
| EMCS | N-[ε-maleimidocaproyloxy]succinimide ester | 9.4 Å |
| GMBS | N-[γ-maleimidobutyryloxy]succinimide ester | 10.2 Å |
| SMCC | Succinimidyl4-[N-maleimidomethyl]-cyclohexane- | 11.6 Å |
| | 1-carboxylate | |
| LC-SMCC | Succinimidyl4-[N-maleimidomethyl]- | 16.1 Å |
| | cyclohexane-1-carboxyl-[6-amidocaproate] | |

Reagents to add thiols on protein amino groups

| | | |
|---|---|---|
| DTSSP | 3,3′-Dithiobis[sulfosuccinimidyl propionate] | 6.8 Å |
| IT | 2-Iminothiolane | 8.1 Å |
| LC-SPDP | Succinimidyl 6-[3-(2-pyridyldithio)- | 15.6 Å |
| | propionamido]hexanoate | |

The modification of amino groups of proteins by either MS reagent or with thiol reagent can be carried out at physiological conditions. Two to three thiols will be generated on nanoparticles and only one maleimide group will be added on albumin. These two products will be mixed in equimolar ratio to add two to three copies of albumin on the surface of nanoparticles. Alternatively, two or three maleimides will be added on nanoparticles and one thiol on albumin to generate a similar conjugate. The reaction conditions, reagent to protein ratio, pH, and incubation time will be manipulated to attain desired level of reactions. The number of thiols added will be estimated by 4-PDS reaction and the maleimide groups will be estimated by the new protocols.

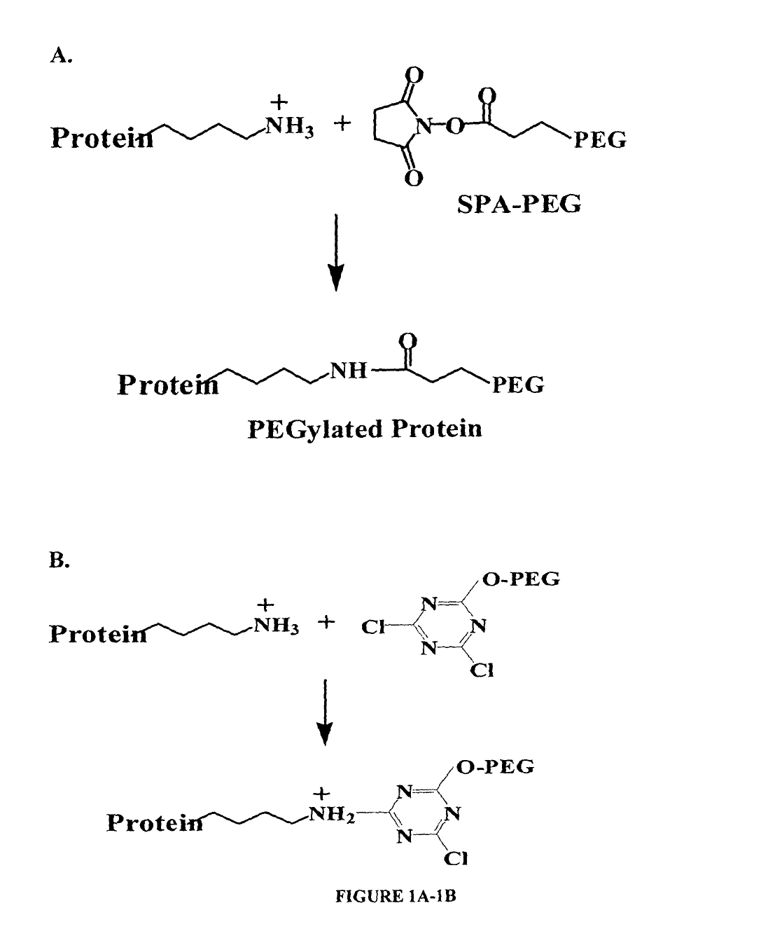

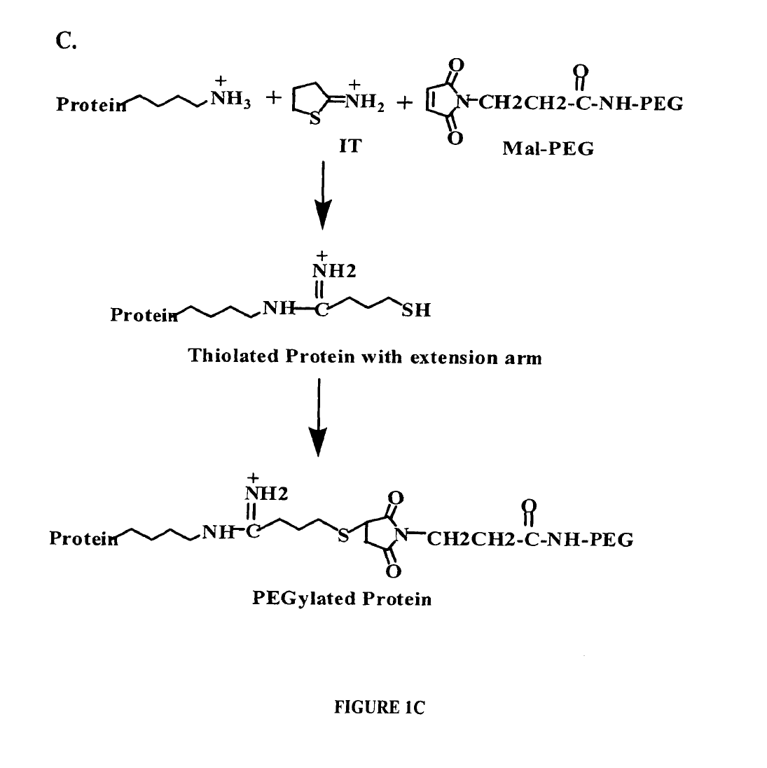

The Extension Arm Facilitated PEGylation (“EAF PEGylation”) involves the modification of protein amino groups by a thiolation reagent, 2-iminothiolane that adds an extension arm carrying a thiol group at the distal end of the proteins amino groups (FIG. 1C). These added thiols can be PEGylated using a thiol specific PEG reagent such as maleimide-PEG. The extension arm carrying a thiol group can be added using any reagent listed in Table 2. Similarly any PEG reagent that can react with thiols such as PEG-iodoacetamide or PEG-vinylsulfone can be used for PEGylation in this protocol.

The EAF PEGylation has been employed to mask the blood group antigens of RBC to generate a universal RBC (12). This protocol masked the antigens better than any PEGylation protocol used in previous investigations. (13-17)

Succinimidyl chemistry-based PEGylation is the most widely used PEGylation to develop therapeutic proteins and peptides (18-20). This protocol also involves modification of the protein surface amino groups (FIG. 1B). However, unlike the EAF PEGylation, this protocol does not add any extension arm on protein amino groups and considered as direct PEGylation. Therefore, PEGylation by this chemistry keeps the conjugated PEG chains closer to the protein surface than the EAF PEGylation and can increase the viscosity of protein better than the EAF PEGylation. Thus, different PEGylation protocols have different advantages.

The EAF PEGylation modifies protein amino groups by imidination and succinimidyl chemistry based PEGylation modifies amino groups by acylation. Therefore, these two protocols may have different site selectivity for the modification of albumin polymer. This can help to modulate the extent of PEGylation of the polymer. Accordingly, these two PEGylation protocols can be used to conjugate PEG chains to albumin polymer. Maleimide-PEG that is used in EAF PEGylation and succinimidyl chemistry based PEG reagents commercially available in different PEG chain lengths varying from 2,000 to 20,000 Da. Using these PEGylation protocols in combination with different size PEG reagents, the degree of PEGylation of albumin polymer will be adjusted to achieve the desired level of viscosity. The extent of PEGylation can also be controlled by modulating the protein to PEG reagent ratio.

Characterization studies: The molecular size of the albumin polymers and their PEGylated versions can be determined by matrix-assisted laser desorption/ionization (MALDI) mass spectrometry. The NMR approach has been employed to determine the number of PEG chains conjugated to albumin molecules (21). The molecular radius of the molecules can be determined by dynamic light scattering measurements. Sodium dodecyl sulfate polyacrylamide (SDS-PAGE) patterns, size exclusion chromatography (SEC) and reverse phase high performance liquid chromatography (RPHPLC) patterns will be determined to evaluate the homogeneity and consistency of the samples for every batch.

Design of albumin polymer: Several protein crosslinking reagents such as glutaraldehyde, disuccinimidyl suberate (DSS), bismaleimidobutane (BMB), and succinimidyl-maleimidomethyl-cyclohexane carboxylate (SMCC) are known. Some reagents are homobifunctional crosslinkers targeted for amino groups or thiol groups and others are heterobifunctional reagents involving both amino and thiol group modification. Glutaraldehyde is a well-noted crosslinker of protein amino groups. This reagent has been used to develop hemoglobin oligomers to use as blood substitutes (22-24). However, these products are heterogeneous in size. Generation of homogeneous protein polymers using any crosslinking reagent has been a challenge.

### EXAMPLES

In an example, 0.5 mM bovine serum albumin was polymerized in the presence of 50 mM DTT in PBS at room temperature for 45 minutes and then half-diluted with 100 mM N-ethyl maleimide to block the remaining free thiols. The size exclusion chromatography of this reaction mixture is displayed in FIG. 1. The polymer has a very homogeneous molecular size. The yield of the polymer is about 50% but can be further improved by adjusting reaction conditions. Additionally, the polymer can be purified to 100% homogeneity by removing the unreacted albumin using size exclusion chromatography. Similar results were obtained with human serum albumin and also at different molar ratios of albumin and DTT.

The polymerization reduced the COP of albumin about 5 fold and enhanced the viscosity about 35% (Table 3). The albumin polymer is PEGylated with SPA-PEG-5000 as a model PEG reagent. This PEG-Alb-polymer is determined to have a COP of 44 mmHg and a viscosity of 8.3 cp at 2.6% protein concentration. Similar results were obtained with human serum albumin. Animal model studies with alginates and dextrans suggest an ideal solution should have a viscosity of approximately 7-10 cp and COP <50 mmHg. Thus, the current PEG-Alb-polymer carries the properties of an ideal plasma expander. A purified albumin polymer on PEGylation is expected to yield a product with much higher viscosity and lower COP.

The stability of the inter-molecular crosslinks of the polymer in plasma was tested by incubating the polymer (about 1%) in plasma (50%) at 37° C. for 24 hours. The sample was analyzed on SEC before and after incubation. No difference in the chromatographic pattern was observed, demonstrating the stability of the polymer in plasma at 37° C. The free thiol content of the polymer is determined to be negligible (much below one).

The PEG-Alb-Polymer did not induce RBC aggregation in normal hamsters when introduced as a 10% blood volume (estimated as 7% of the body weight) hypervolemic bolus infusion. These results, demonstrate the PEG-protein polymerization chemistry. Characteristics can be modified to make it suitable as a super plasma expander.

### 2. Results

The invention provides new PEG-albumin polymer-based plasma expanders with ideal solution properties that can be used to extend the transfusion trigger, delaying the use of blood transfusions, especially in critical conditions such as combat casualties, highway accidents and causalities in remote areas. These polymers can also be used for efficient delivery of drugs, particularly anticancer drugs to tumors.

The COP and viscosity of a solution containing 50% HSA polymer (Alb-polymer-50) are displayed in Table 3. The polymerization reduced the COP of albumin about 5 fold and enhanced the viscosity about 35%.

TABLE 3
Viscosity and COP of albumin samples

| | Protein concentration | COP | Viscosity |
|---|---|---|---|
| Sample | (mg/ml) | (mmHg) | (cp) |
| Albumin | 16 | 8.2 | 0.9 |
| Alb-Polymer-50 | 16 | 1.5 | 1.2 |
| PEG-Alb-Polymer-50 | 26 | 44 | 8.3 |
| PEG-Albumin | 25 | 38 | 2.7 |

The degree of PEGylation can be controlled by adjusting the protein to PEG reagent ratio. The pattern of PEGylation can to be manipulated by using different size PEG reagents. The same amount of PEG can be conjugated to a protein molecule in different ways. For example a total of 20,000 PEG units can be added by conjugating only one PEG chain of 20 kDa or 2 chains of 10 kDa or 4 chains of 5 kDa. In this way, degree and pattern of PEGylation can be modulated.

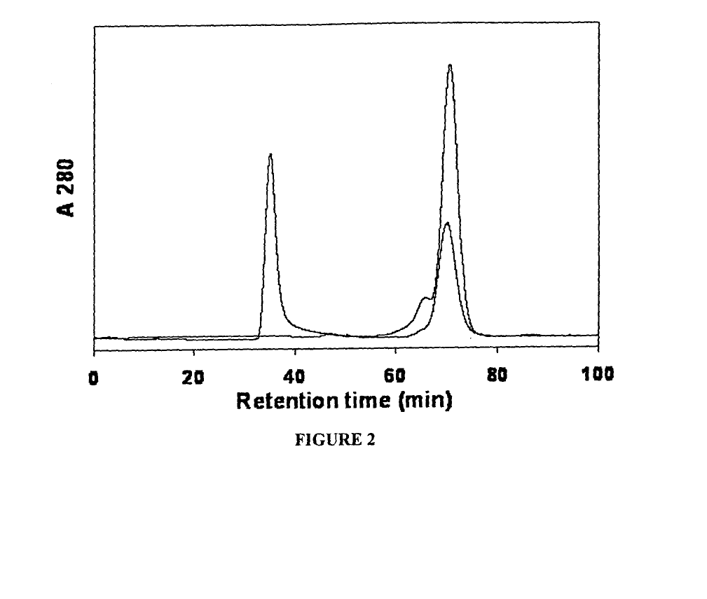

An albumin polymer has been generated employing thiol group crosslinking. The size exclusion chromatogram (SEC) of the polymer is shown in FIG. 2. This polymer has a very homogeneous size. The polymer can be isolated to 100% purity by removing the unreacted albumin using size exclusion chromatography.

The PEGylated-Alb-polymer that is generated carries a COP of 44 mmHg and a viscosity of 8.3 cp at 2.6% protein concentration (Table 3). Animal model studies with alginates and dextrans suggest an ideal solution should have a viscosity of approximately 7-10 cp and COP <50 mmHg (8, 9, 25). Thus, the current PEGylated-Alb-polymer carries the properties of an ideal plasma expander. Studies indicate that this PEGylated-Alb-polymer does not induce red blood cell (RBC) aggregation even at normal hematocrits.

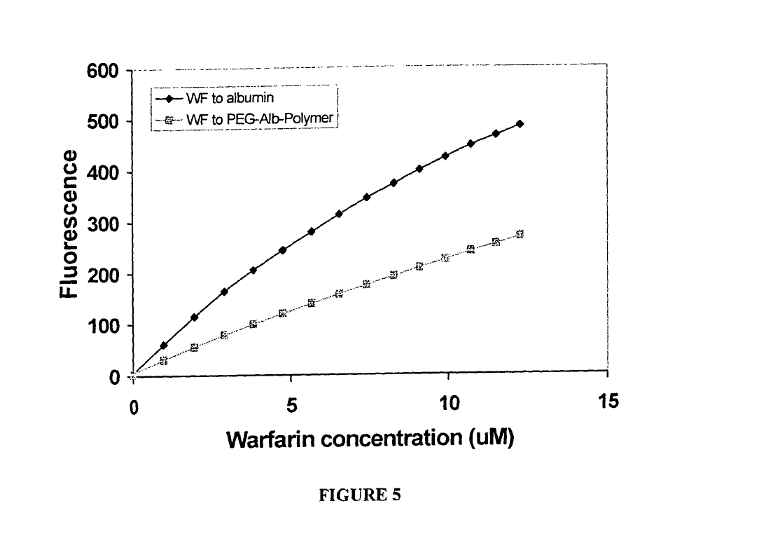

The drug binding capability of PEG-albumin-polymer is studied using warfarin as a model drug. Warfarin is an anticoagulant that binds to albumin at site-1 binding site. It is a fluorescent molecule and its fluorescence increases on binding to albumin. The binding of this drug to albumin has been reduced on polymerization and PEGylation (FIG. 5). The drug binding capability of the PEG-albumin-polymer is only 50% of that of albumin. However, the enhanced circulation life of the PEG-albumin-polymer can easily compensate its reduced drug binding capability and can improve the pharmacokinetics of the attached drugs.

### 3. Discussion

Albumin is a molecule with almost identical physical configuration as Hb, but lacking the potentially toxic heme. Microvascular function in extreme hemodilution was significantly improved with PEG-Alb over the improvement attained with PEG-Hb. A problem is the high oncotic pressure (COP) of these PEG compounds impedes achieving high plasma concentrations (8). The volume of the extracellular fluid exchanged, due to the COP of the solutions, is determined primarily by the colloidal concentration of each fluid. However, the dissimilarities in results between PEGylated proteins and conventional colloids suggest the presence of other mechanisms probably related to the structural modification on the protein by PEGylation. Albumin solution has a uniform molecular size (monodisperse). PEGylated Alb-theoretically remains in the intravascular compartment for a longer time than the unPEGylated albumin, providing larger and long lasting plasma volume expansion for identical infused volumes.

The objective of conjugating PEG chains to therapeutic molecules is to extend the circulation life of the therapeutics (26, 27). PEG chains are hydrophilic, get heavily hydrated and can cover a large surface area of the proteins. Extension of circulation life of PEGylated molecules may be due to several reasons; reduced susceptibility to enzyme hydrolysis, camouflaged from the host immune system and reduced renal clearance due to enhanced molecular size. In addition to extending the circulation life of protein, PEGylation also enhances the COP and viscosity of the conjugated proteins (28, 29). Although high viscosity is desired for an optimal plasma expander, the parallel increase of COP negates the effect by causing diffusion of interstitial fluid into vasculature thus reducing the plasma viscosity. A goal of the present plasma expander is to have high viscosity and a low COP so that when infused the plasma viscosity can be increased to around 2 cp and the hematocrit as high as 30%.

### As Super Plasma Expander for Enhanced Plasma Expansion for Recovery of Microvascular Function

The current invention involves the design of PEGylated-albumin polymers (human and bovine serum albumin (Alb)) carrying high viscosity and low colloid osmotic/oncotic pressure (COP) to serve as optimal plasma expanders. The polymers of albumin are generated inducing the polymerization using reducing agents such as DTT and TCEP. These reagents dissociate the intrinsic disulfide bonds of albumin generating free thiols. Since the unfolded structure is unstable, the free thiols inherently form new disulfide bridges, inter- and intra-molecular cross-linking, leading to polymerization of albumin.

TABLE 4
Solution Properties of Plasma Expanders

| | | | |
|---|---|---|---|
| Plasma Expanders | MW | Viscosity | COP |
| concentration, % | kDa | cP | mmHg |

High Viscosity PE

| | | | |
|---|---|---|---|
| Alginate 0.7 | 450-1200 | 8.0 | 0 |
| Dex 500 6 | 500 | 6.5 | 32 |

Moderate Viscosity PE

| | | | |
|---|---|---|---|
| HES 6^{a} | 550 | 3.4 | 29 |
| HES 10^{b} | 200 | 3.0 | 85 |
| Dex 70 6 | 70 | 2.8 | 50 |
| PEG-Alb 2.5^{c} | 126 | 2.7 | 38 |
| MPA, PEG-Alb 4^{d} | 96 | 2.2 | 48 |
| HSA 10 | 66 | 1.5 | 47 |

Low Viscosity PE

| | | | |
|---|---|---|---|
| HSA 5 | 66 | 0.9 | 21 |
| RL | — | 0.8 | 0 |

Dex500, dextran 500 kDa;
HES, Hydroxyethyl Starch;
Dex70, dextran 70 kDa;
PEG-Alb, polyethylene glycol conjugated albumin;
MPA, PEG-Alb 4%;
HSA, human serum albumin;
RL, Ringer's lactate.
^{a}Hextend ®, BioTime, Berkeley, CA.
^{b}Pentaspan ®, B. Braun Medical, Irvine, CA,
^{c}supplied by Dr. Acharya, Albert Einstein College of Medicine, Bronx, NY;
^{d}Sangart, San Diego, CA.

PEGylated albumins generated served as excellent plasma expanders in hemorrhagic shock and endotoxemia induced hamster models. They were found better than current conventional expanders (21, 30) because they did not induce RBC aggregation. However, they are not completely ideal since these products are associated with high oncotic pressure, and thus their ability to increase plasma viscosity in vivo is limited. Increasing molecular size by polymerization of albumin prior to PEGylation will increase viscosity without a concomitant increase in oncotic pressure.

High plasma viscosity in anemia (increasing from 1.2 cp normal to approximately 2.0 cp) is the critical factor in maintaining microvascular function. Microvascular function has been defined as FCD and has been shown to be more highly correlated to outcome/survival than oxygen delivery during shock (6). However, currently available plasma expanders were designed to recover blood pressure, but do not address microvascular dysfunction.

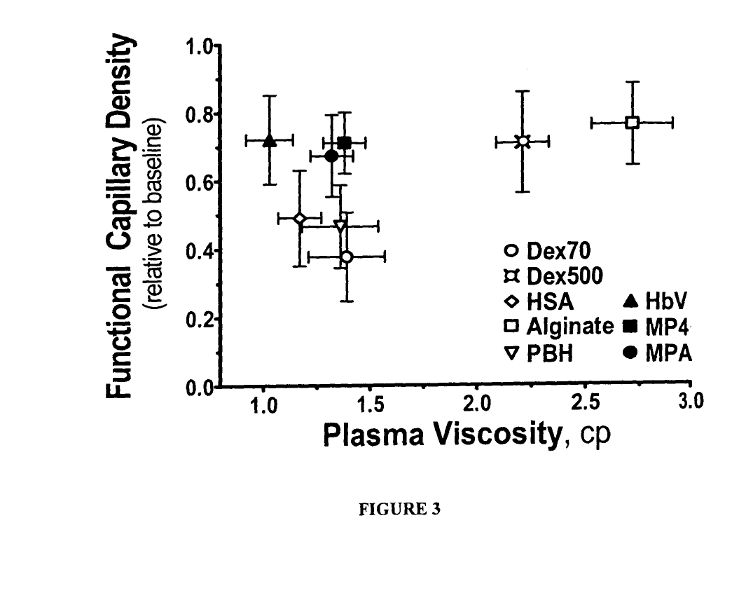

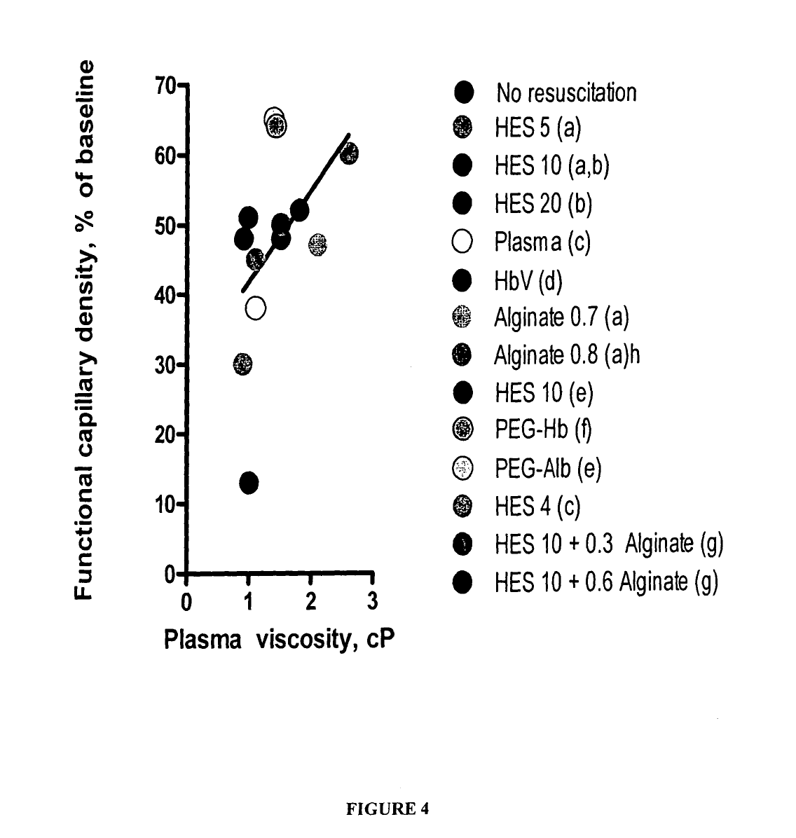

The relationship between FCD and viscosity during extreme hemodilution and hemorrhagic shock resuscitation are shown in FIGS. 3 and 4, respectively. Dextrans and alginate molecules > 1,000 kDa increase plasma viscosity and maintain FCD significantly better. PEG-protein solutions (PEG-Alb and PEG-Hb) also can maintain FCD despite not increasing plasma viscosity. Highly viscous fluids (alginate and high molecular weight dextrans) can be used only at comparatively low hematocrits (< 18%) because a higher concentration of RBC leads to red blood cell aggregation. Conjugation of albumin to make equivalently large polymers combined with the beneficial effects attributable to the conjugation with PEG can be used to obtain the desired increase in plasma viscosity to ˜2.0 cp. These PEG-Alb-polymers do not cause RBC aggregation even at normal hematocrits.

PEGylation is known to reduce immunological response and the PEG molecule's ability to retain water gives it the ability to eliminate any potential difficulties associated with RBC aggregation. Nacharaju et al. have shown that the extent of camouflage of RBC antigens by PEGylation is dependent on the efficiency of the PEGylation protocol employed (12, 31). Addition of an extension arm on RBC membrane protein amino group increases the accessibility of the site for PEGylation. Using this approach, an efficient masking of RBC antigens has been achieved and the agglutination of RBCs by the respective antibodies has been inhibited completely. PEGylation of vesicles (200 nm diameter) has also eliminated RBC aggregation (32). PEGylation of albumin polymer with this protocol can help to prevent the potential RBC aggregation.

Accordingly, PEGylated-Alb-polymers can restore microvascular function from hemorrhagic shock, extreme hemodilution and endotoxemia and extend the transfusion trigger, delaying the use of blood transfusions. Unlike current plasma expanders, the PEG-albumin polymers designed in the current invention have extended circulation life, minimal side effects and are superior to the existing plasma expanders. Therefore, The PEGylated-albumin polymers can be used as plasma expanders for routine clinical conditions and also to extend transfusion trigger in critical conditions such as combat casualties, highway accidents and causalities in remote areas where blood transfusion is not readily available. Since these products have enhanced molecular size these can be used to treat complications associated with capillary leak such as sepsis.

### As Nanoparticles for Drug Delivery

Albumin polymers and PEG-Alb polymers with a molecular size of between 60 and 80 nm (Table 1) can be used as carriers for therapeutic agents for prolonged circulation. Albumin receptors are widely distributed in body (liver, lungs, intestine etc). Receptor mediated targeted drug delivery is possible with these polymers. Albumin bound drugs for hepatitis-C and cancer therapies are found to have improved efficacy and safety compared with conventional drugs. The PEG-Alb-polymers can be used in more efficient therapeutics. The current invention does not use any potential toxic agents for the preparation of albumin nanoparticles. Thus, side effects are expected to be much lower.

Albumin nanoparticle bound paclitaxel (Abraxane®) is a new drug approved for the treatment of recurrent breast cancer. Albumin conjugated interferon (Albuferon) is under phase trials for hepatitis-C therapy.

Nanotechnology is a new field of interdisciplinary research that has expanded rapidly and widely over the last few years to help overcome problems in medicine. Nanoparticles extend circulation life of therapeutic molecules. There are many examples of the development of this discipline, with tools applicable to different diseases. Most well studied are liposomes, dendrimers, super paramagnetic nanoparticulates, polymer-based platforms, gold nanoshells, silicon- and silica-based nanoparticles carbon-60 fullerenes, and nanocrystals.

Protein based nanoparticles (“NP”) are biodegradable and hence interest for such nanotechnology is increasing. Albumin nanoparticles are particularly preferable since albumin is a plasma protein and can camouflage the albumin bound therapeutic molecules from the immune system efficiently. This will help to increase the circulation life of the carried drugs and lower the induction of immune response (antibodies development). The preferential uptake of albumin in tumors and inflamed tissue, ready availability, biodegradability, and lack of toxicity and immunogenicity makes albumin preferential candidate for drug delivery. Moreover, albumin receptors are widely distributed in body such as liver, lungs and intestine. Therefore, receptor mediated targeted drug delivery is possible. Albumin has two high affinity drug binding sites. Thus, non-covalent binding of drugs as in the case of Abraxane and covalent attachment of drugs as in Albuferon is feasible. A combination therapy with more than one drug employing covalent and non-covalent interactions is also achievable.

Albumin nanoparticles used as drug carriers such as Taxanes, in particular the currently available paclitaxel (Taxol®; Bristol-Myers Squibb Co, Princeton, N.J., USA) and docetaxel (Taxotere®; Aventis Pharmaceuticalslnc, Bridgewater, N.J., USA), represent an important class of antitumor agents which have proved to be fundamental in the treatment of advanced and early-stage breast cancer. Both these drugs are included in the treatment regimens for adjuvant chemotherapy and are indicated as preferred agents for recurrent and metastatic breast cancer by The National Comprehensive Cancer Network (NCCN) clinical practice guidelines for breast cancer (National Comprehensive Cancer Network, Clinical Practice Guidelines in Oncology: Breast Cancer v2, 2008. Available at www.nccn.org/professionals/physician_gls/default.asp.). Albumin nanoparticle bound-docetaxel and rapamycin are currently in early clinical trials.

Comparison of albumin nanoparticles of the current invention and earlier version: the albumin nanoparticles used in Abraxane seem to be generated by glutaraldehyde mediated cross-linking of albumin amino groups (Glu-Alb-NP). Protein amino groups are the most widely used functional groups for the conjugation of PEG and/or drugs. Most the cross-linking reagents commercially available are targeted to amino groups. Glutaraldehyde cross-linking of albumin uses surface amino groups. Therefore, limited number of amino groups are available for tagging PEG or drugs to Glu-AIb-NP. Besides, Glu-Alb-NP are generated by desolvation process employing ethanol before crosslinking with glutaraldehyde. Therefore, these nanoparticles are highly hydrophobic and may be useful to carry only hydrophobic drugs such as taxanes.

While earlier albumin nanoparticles (Glu-Alb-NP) were glutaraldehyde cross-linked with many blocked amino acids, insoluble, could be toxic, and had no PEG (could only be hydrophobic), the current albumin nanoparticles (DTT-Alb-NP) are intrinsically thiol cross-linked with free amino acids, solubility can be customized, is unlikely to be toxic, and is PEGylated with customizable hydrophobicity. The current version albumin nanoparticles are prepared by the cross-linking of intrinsic thiols of albumin mediated by DTT (DTT-Alb-NP). Therefore, the surface amino groups of albumin are available for further derivatization. Thus, drug carrying capacity will be higher for these NP.

The solubility of DTT-Alb-NP varies with the extent of polymerization. The polymers (nanoparticles) partition into insoluble phase as the polymerization proceeds. Thus, by controlling the degree of polymerization, the particle size and solubility of the polymers can be adjusted. These parameters can be further customized by PEGylation. Soluble form of nanoparticles may have advantages in the circulation and internalization of drugs into the targeted sites/cells.

It is likely that the albumin polymers generated in the current invention are not recognized by albumin receptors for targeted drug delivery. The nanoparticles can be surface decorated with albumin monomers for the receptor mediated recognition.

Interferons (IFNs) are widely being used in antiviral and anti-cancer therapy. Several IFN based drugs are approved for antiviral and anticancer therapies. IFN in combination with Ribavirin is a standard therapy for hepatitis-C. Due to the high clearance rate of IFN, the treatment involves frequent dosage (administered subcutaneously), three times per week over 24-48 weeks, depending upon the genotype of the virus.

Pegasys® (PEG-40 kDa-interferon alpha-2a, Hoffmann La Roche, N.J.) and PEG-Intron (PEG-12 kDa-interferon alpha-2b, Schering Plough (now Merck, Whitehouse Station, N.J.) are two PEG conjugated IFN drugs currently in use for hepatitis-C therapy. Since PEGylation reduces the clearance rate these drugs are taken only once a week subcutaneously, in combination with ribavirin (orally) everyday. Un-PEGylated and PEGylated IFNs induce the production of antibodies to IFN and leads to discontinuation of treatment for some patients. Albuferon (Human Genome Sciences) is a new drug under phase trial for Hepatitis-C therapy. It is a genetically fused conjugate of albumin and IFNa-2b. The carboxyl end of albumin is linked to the amino end of IFN.

The therapeutic efficiency of the IFN drugs seems to correspond to in vivo clearance rate rather than the actual bioactivity. The drop in the bioactivity of IFN due to the conjugation to PEG or albumin is compensated by the extended circulation life of the drugs. The longer circulation life of Albuferon than PEGylated interferons may be a consequence of its higher molecular size. Alternatively, albumin may give better protection to the conjugated molecules from the immune system than PEG. Consistent with this, the induction of anti-IFN antibodies is less frequent with Albuferon (33). In addition, albumin receptor mediated delivery of Albuferon to liver could be responsible for its higher therapeutic efficiency.

Albuferon is a 1:1 complex of albumin and IFN. Its molecular weight is 87 kDa and its hydrodymanic radius is expected to be less than 10 nm. Conjugation of IFN to PEG-albumin polymer can further extend the circulation of IFN and hence can improve its therapeutic potency. Albuferon is a genetically fused complex of albumin and IFN. Conjugation of IFN to the current albumin nanoparticles by chemical methods may be easier and cheaper than genetic fusion. Another advantage is multiple copies of IFN can be conjugated to the nanoparticles for higher potency of drug.

Albumin is a major component of plasma with drug binding capabilities which makes it a natural plasma expander and efficient therapeutic carrier. Nanoparticles of albumin with enhanced size, viscosity and circulation life can serve as a better plasma expander as well as a more efficient drug carrier.

### REFERENCES

1. R. M. Winslow, Semin Hematol 44, 51 (Jan, 2007).
2. A. I. Alayash, Nature Reviews Drug Discovery 3, 152 (2004).
3. P. W. Buehler, A. I. Alayash, Biochim Biophys Acta 1784, 1378 (2008).
4. B. D. Spiess, J Appl Physiol 106, 1444 (2009).
5. E. Tsuchida et al., Bioconjug Chem (2009).
6. H. Kerger, D. J. Saltzman, M. D. Menger, K. Messmer, M. Intaglietta, Am J Physiol 270, H827 (1996).
7. K. D. Vandegriff, R. M. Winslow, Artif Organs 33, 133 (2009).
8. A. S. Acharya, M. Intaglietta, A. G. Tsai, Transfusion Alt in Transfusion Med 9, 254 (2007).
9. P. Cabrales, J. Martini, M. Intaglietta, A. G. Tsai, Am J Physiol 291, H581 (2006).
10. A. G. Tsai, B. Friesenecker, M. McCarthy, H. Sakai, M. Intaglietta, Am J Physiol 275, H2170 (1998).
11. A. G. Tsai et al., Am J Physiol Heart Circ Physiol 288, HI730 (2005).
12. P. Nacharaju, F. N. Boctor, B. N. Manjula, and S. A. Acharya, Transfusion 45, 374 (2005).
13. D. P. Blackall, J. K. Armstrong, H. J. Meiselman, and T. C. Fisher, Blood 97, 551 (2001).
14. A. J. Bradley, K. L. Murad, K. L. Regan, and M. D. Scott, Biochim Biophys Acta 1561, 147 (2002).
15. T. C. Fisher, and J. K. Armstrong, in U.S. Pat. No. 6,312,685 (-2001).
16. M. D. Scott, K. L. Murad, F. Koumpouras, M. Talbot, and J. W. Eaton, Proc Natl Acad Sci USA 94, 7566 (1997).
17. S. Mathur, B. Clark, G. Castro, X. M. Zhou, S. Bowers, and A. Stassinopoulos, Blood 102, 556A (2003).
18. C. D. Conover, R. Linberg, C. W. Gilbert, K. L. Shum, R. G. Shorr, Artif Organs 21, 1066 (1997).
19. S. Frokjaer, D. E. Otzen, Nat Rev Drug Discov 4, 298 (2005).
20. S. Foser et al., Protein Expr Purif 30, 78 (2003).
21. P. Cabrales et al., Shock 24, 66 (2005).
22. S. A. Gould, G. S. Moss, World J Surg 20, 1200 (1996).
23. C. D. Conover, R. Linberg, L. Lejeune, M. Nagy, K. L. Shum, Artif Organs 23, 1088 (1999).
24. C. D. Conover et al., Artif Cells Blood Substit Immobil Biotechnol 27, 93 (1999).
25. P. Cabrales, A. G. Tsai, M. Intaglietta, Resuscitation 77, 379 (2008).
26. J. M. Harris, N. E. Martin, M. Modi, Clin Pharmacokinet 40, 539 (2001).
27. M. Werle, A. Bernkop-Schnurch, Amino Acids 30, 351 (2006).
28. S. A. Acharya et al., Artif Cells Blood Substit Immobil Biotechnol 33, 239 (2005).
29. B. N. Manjula et al., Protein J 24, 133 (2005).
30. N. Hangai-Hoger et al., Crit Care Med 34, 108 (2006).
31. P. Nacharaju, B. N. Manjula, S. A. Acharya, Artif Cells Blood Substit Immobil Biotechnol 35, 107 (2007).
32. H. Sakai et al., J Biomed Mater Res 40, 66 (1998).
33. Harrison, S. A., 2007, Am J Gastroenterol 102, 2332-2338

1. A process for preparing an albumin polymer, the method comprising contacting albumin with a reducing agent under conditions causing dissociation of intrinsic albumin inter-molecular disulfide bridges and subsequently permitting crosslinking of the albumin by formation of new inter-molecular and intra-molecular disulfide bridges, so as to form the albumin polymer.

2. The process of claim 1, wherein the reducing agent is dithiothreitol or tris(2-carboxyethyl)phosphine.

3. The process of claim 1, further comprising contacting the albumin polymer with a derivatized polyethylene glycol (PEG) under conditions permitting formation of a bond between the PEG and the albumin polymer.

4. The process of claim 3, wherein the derivatized PEG is succinimidyl-PEG, cyanuric chloride-PEG or maleimide-PEG,

5. The process of claim 3, further comprising purifying the albumin polymer by size-exclusion chromatography prior to PEGylating the albumin polymer.

6. An albumin polymer comprising one or more non-intrinsic crosslinking inter-molecular and intra-molecular disulfide bridges.

7. A process for preparing a PEGylated albumin polymer, the method Comprising contacting an albumin polymer with a derivatized polyethylene glycol (PEG) Under conditions permitting formation of a bond between the PEG and the albumin Polymer so as to form a PEGylated albumin polymer.

8. The process of claim 7, wherein a reducing agent is used to dissociate one or more intrinsic disulfide bonds of the albumin before polymerizing the albumin.

9. The process of claim 7, wherein the reducing agent is dithiothreitol or tris(2-carboxyethyl)phosphine.

10. The process of claim 7, the method further Comprising separating the polymerized albumin from unreacted albumin before PEGylation.

11. The process of claim 10, wherein the polymerized albumin is separated by size exclusion chromatography.

12. The process of claim 7, wherein the derivatized PEG is succinimidyl-PEG, cyanuric chloride-PEG or maleimide-PEG.

13. The process of claim 7, wherein the method Of PEGylating the albumin polymer comprises:
a) contacting the albumin polymer with a thiol agent; and
b) contacting the product of step a) with maleimide-PEG,
so as to thereby form a PEGylated albumin polymer.

14. The process of claim 7, further comprising bonding at least one albumin monomer to the surface of the PEGylated albumin polymer.

15. The process of claim 14, wherein the bonding of at least one albumin monomer to the surface of the PEGylated albumin polymer is effected through a maleimide-thiol reaction.

16. A PEGylated albumin polymer prepared by the process of claim 7.

17. The PEGylated albumin polymer of any of claim 16, wherein the PEGylated albumin polymer has a hydrodynamic radius of between 25 and 200 nm.

18-22. (canceled)

23. The PEGylated albumin polymer of claim 16, wherein the PEGylated albumin polymer has a colloid osmotic pressure between 0 and 60 mm Hg at 2.6% protein concentration.

24-25. (canceled)

26. A pharmaceutical composition comprising a therapeutically effective amount of the PEGylated albumin polymer of claim 16, in a pharmaceutically acceptable carrier.

27-29. (canceled)

30. A method of treating blood loss in a subject, the method comprising administering to the subject the PEGylated albumin polymer of claim 26, in a therapeutically effective amount so as to treat the blood loss.

31-37. (canceled)
